(19) United States
(12) Patent Application Publication
(10) Pub. No.: US 2008/0045893 A1
(43) Pub. Date: Feb. 21, 2008

(54) Imageable balloon and method of making

(75) Inventors: Michael F. Aita, Milwaukee, WI (US); Milan Mursec, Milwaukee, WI (US)

Correspondence Address: GODFREY & KAHN S.C., 780 NORTH WATER STREET, MILWAUKEE, WI, 53202, US

(73) Assignee: Cardio Exodus, LLC

(21) Appl. No.: 11/354,589

(22) Filed: Feb. 14, 2006

(51) Int. Cl. A61M 29/00; B32B 37/00

(52) U.S. Cl. 604/96.01; 156/272.2; 604/103.01

(57) ABSTRACT

A method of making a remotely imagable balloon for use with a balloon catheter, such as might be used for angioplasty procedures. Adhesive and imagable material may be coated onto an inner surface of the balloon before assembly into a balloon catheter and the adhesive may be allowed to cure and fix the imagable material within the balloon. A powder injector for inserting imagable material within a balloon. A curing station for curing light cured adhesive coated along an inner surface of a balloon.

### FIELD

The present invention relates generally to methods and apparatus for making imagable angioplasty balloons.

### BACKGROUND

Stenosis of vascular elements in humans can pose a severe health risk, particularly when coronary vessels are involved. A variety of methods of addressing and resolving stenosis problems have been implemented to varying degrees of success. Once of the more acceptable and at least moderately successful methods of addressing stenosis of coronary vessels has been the use of angioplasty. Angioplasty generally refers to a method of compressing the material occluding the coronary vessel to reopen the vessel, permitting increased blood flow past the occlusion.

Angioplasty is conventionally carried out with a catheter inserted at a site distant from the occlusion, where the catheter includes an expandable distal end portion, generally referred to as a balloon. At the time of insertion, the balloon is preferably in a collapsed or unexpanded compact shape. The catheter is advanced from the insertion site to the site of the occlusion so that the balloon is inserted through the occlusion. The balloon is then expanded, either mechanically or by the injection of a fluid through a lumen of the catheter which inflates the balloon. As the balloon expands, the material forming the occlusion is compressed against the walls of the vessel. After the balloon is deflated or recompacted, the material forming the occlusion remains at least partially in a compressed state against the vessel wall, providing a larger opening through the vessel at the site of the occlusion.

While a variety of approaches exist for identifying the location and extent of occlusion within the vasculature of a patient, one of the keys to successful angioplasty is the correct positioning of the distal end of the catheter with respect to the occlusion to be addressed. One of the approaches that may be used to locate occlusions and catheters within a patient's vasculature involves the use of radio imaging. Unfortunately, many of the materials used to construct angioplasty catheters and balloons are generally radiotransparent, meaning that the distal end of the catheter may not be generally visible to an operator of a radio imaging system.

It is also desirable to have the balloon or distal end of the angioplasty catheter be radiopaque so that the extent to which the occlusion is opened and blockage removed can be verified. Conventional approaches to this desire for visibility of the inflated balloon were addressed by the use of a radiopaque fluid to inflate the balloon during compression of the occlusion. However, such radiopaque fluids can be quite viscous and require a relatively large lumen be included in the catheter to permit injection of the fluid through the catheter to inflate the balloon. The need for large lumens within the catheter can lead to the overall external diameter of the angioplasty being undesirable large, and may limit the size of the vessels within which occlusions can be treated.

Conventional methods have provided a variety of constructions of catheters and balloons to insert or include radiopaque material in or around the distal end of angioplasty catheters. One of these approaches is described in commonly owned and invented U.S. Pat. No. 6,884,234, where radiopaque material is added to an inner surface of the balloon, without hindering the expansion or contraction of the balloon. Constructing catheters and balloons which include such radiopaque materials may be difficult to accomplish and therefore may result in a higher cost of manufacture.

Improvements to conventional methods of manufacturing radiopaque balloons are desirable.

### SUMMARY

The present invention relates generally to a method of making a remotely imagable balloon for use with a balloon catheter, such as might be used for angioplasty procedures. Adhesive and imagable material may be coated onto an inner surface of the balloon before assembly into a balloon catheter and the adhesive may be allowed to cure and fix the imagable material within the balloon. The present invention also relates to a powder injector for inserting imagable material within a balloon. The present invention further relates to a curing station for curing light cured adhesive coated along an inner surface of a balloon.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a diagrammatic view of a balloon coating apparatus according to the present invention.

FIG. 2 is a closer view of the powder injector of FIG. 1.

FIG. 3 is a view of a portion of an electrostatic charging element that may be incorporated into the coating apparatus of FIG. 1.

FIG. 4 is a diagrammatic view of a balloon curing apparatus according to the present invention.

### DETAILED DESCRIPTION OF THE INVENTION

Reference will now be made in detail to exemplary aspects of the present invention which are illustrated in the accompanying drawings. Wherever possible, the same reference numbers will be used throughout the drawings to refer to the same or like parts.

Conventional balloon catheters used in angioplasty have used a variety of approaches and methods to provide a radiopaque or other imagable quality to aid in the positioning of the balloon. One such approach is disclosed in commonly owned and invented U.S. Pat. No. 6,884,234, the disclosure of which is incorporated herein by reference. While the approach of the catheter according to the invention of this patent does permit a uniform coating of an interior surface of the balloon, the process and approach to including the radiopaque material within the balloon could be improved. Particularly, the coating within the balloon may be segmented to aid in compaction of the balloon for insertion. Such segmenting of the radiopaque or imagable coating permits the desirably small compacted size to be achieved but may require a complex manufacturing process.

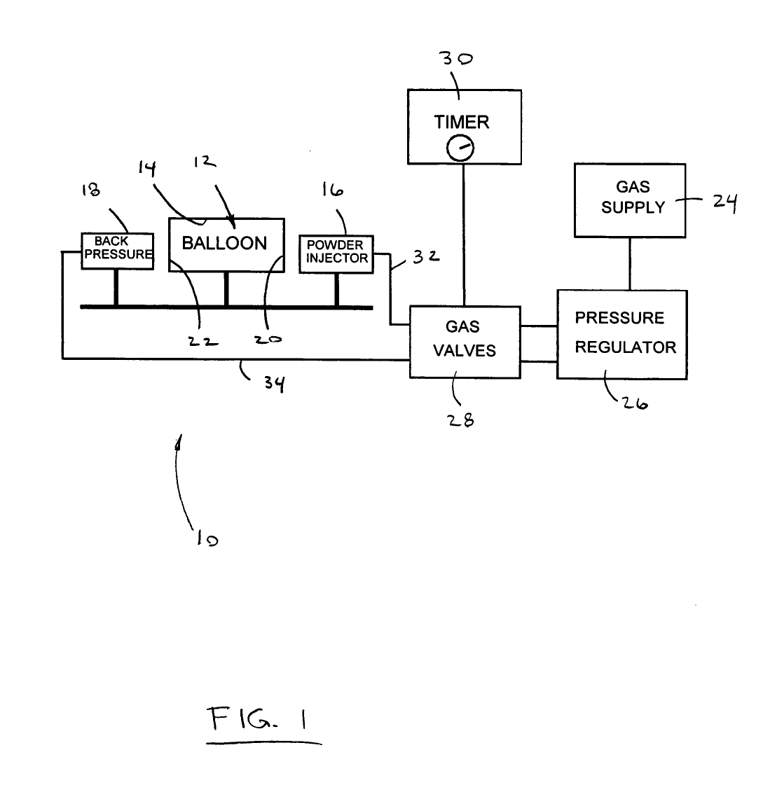

The present invention relates to an apparatus and a method of manufacturing and assembling of balloon catheters that include a uniform radiopaque interior coating and are compactable for insertion through desirably sized guide catheters into a patient's vascular system. An apparatus 10 for coating balloons with radiopaque material according to the present invention is shown diagrammatically in FIG. 1, with a open ended balloon 12 positioned to receive the powder coating. Prior to being positioned within apparatus 10, balloon 12 may be coated along an inner surface 14 with an adhesive to adhere the radiopaque material to the inner surface. Adhesives commonly used for this sort of application tend to be relatively viscous and may need to be thinned prior to application within balloon 12. Methods and devices for coating inner surface 14 are conventionally known and may involve the use an adhesive thinned with a volatile solvent to a desired low viscosity. This adhesive and solvent combination may be injected within balloon 12 while balloon 12 is rotated or otherwise manipulated to ensure that adhesive is applied to inner surface 14 as desired. Once the adhesive has been applied to the desired portions or all of inner surface 14, the solvent used to dilute the adhesive may be allowed to evaporate, leaving a desired coating of adhesive on inner surface 14 and preparing balloon 12 for insertion within coating apparatus 10.

The adhesive may preferably require a separate curing process so that maximum flexibility in the positioning of radiopaque material within balloon 12 is retained, although self curing or air curing adhesives may also be used. Suitable adhesives may include but are not limited to epoxy, urethane, silicone, acrylic, or cyanoacrylate adhesives and corresponding solvents for each adhesive. Corresponding solvents may include but are not limited to acetone, alcohol, freon, or a combination of these solvents.

In the illustrated embodiment, radiopaque material is preferably a powder when injected within balloon 12, and apparatus 10 is configured for injecting and positioning a dry powder within balloon 12. When inserted within apparatus 10, balloon 12 may be engaged by a powder injector 16 at a first open end 20 and a back pressure device 18 at a second opposite open end 22. Connected to each of injector 16 and back pressure device 18 are a gas source 24 and a pressure regulator 26 which are configured to deliver gas at a desired pressure and flow rate. One or more valves 28 are situated between pressure regulator 26 and injector 16 and back pressure device 18 to control flow of gas. Valves 28 may be connected to and actuated by a timer 30. It is preferable that a gas supply line 32 between valves 28 and powder injector 16 be controllable separately from a gas supply line 34 between valves 28 and back pressure device 18, such as through separate valves.

With the adhesive coated and uncured on inner surface 14 of balloon 12 positioned as shown within apparatus 10, a process for positioning radiopaque material within balloon 12 can be carried out. To position the radiopaque material throughout inner surface 14 of balloon 12, pulses of gas through injector 16 and back pressure device 18 may be directed into balloon 12. The gas used for these pulses or bursts is preferably a dry gas, so that the characteristics of the powder in injector 16 may be controlled and maintained without unwanted humidity. These bursts of gas directed by valves 28 through injector 16 and back pressure device 18 are preferably controlled by solenoid-actuated valves. Such valves may permit precise control of the time and duration of gas supplied to injector 16 and back pressure device 18 to ensure uniform and complete coating of inner surface 14. In place of the back pressure device and the injection of gas through second end 22 of balloon 12, a second injector 16 could be positioned at second end 22 and radiopaque material could be injected into both ends of balloon 12. In this way, the gas carrying the radiopaque material into balloon 12 from one end can serve as the back pressure for the radiopaque material being injected through the opposite end.

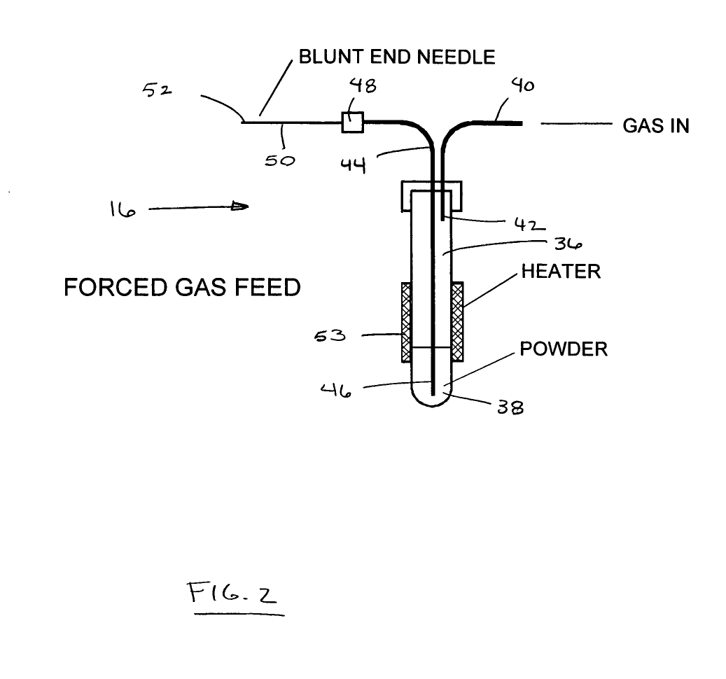

It is preferable that a powdered form of a desired radiopaque material 38 be held within a powder vessel 36 of injector 16, as shown in FIG. 2. Connected to gas supply line 32 in FIG. 1 is a gas inlet 40 with an inner end 42 releasing gas into vessel 36 preferably located near a top end of vessel 36 with powdered material 38 typically adjacent a bottom end of vessel 36. A gas outlet 44 provides a path for material 38 to exit vessel 36 through an inner end 46. An outer end 48 of gas outlet 44 may have a needle 50 or some other appropriate nozzle arrangement for injecting material 38 into balloon 12. An outer end 52 of needle 50 may be positioned at one location within balloon 12 while material 38 is being injected or may be longitudinally moved within balloon 12 to provide a more uniform coating of inner surface 14. The size of needle 50 may be selected based on the size of balloon 12 and the characteristics of radiopaque material. Preferably, needle 50 will range in size from 18 to 25 gauge.

It is desirable that the physical properties of material 38 be maintained as consistent as possible to ensure uniformity of injection and coating. Powdered material 38 may have better flow characteristics when keep as dry as possible, so a heater 53 may be positioned on an exterior of vessel 36 to heat powder 38 and dry off as much water or other moisture as possible prior to injection. It is anticipated that material 38 may have flow characteristics which do not require such preheating or may not be susceptible to atmospheric moisture, so that a heater is not required as part of injector 16. To improve flow of material 38 through injector 16, injector 16 may also be physically agitated before material 38 is injected into balloon 12 to aerate material 38. Injector 16 may also include an integral mechanism such as a vibrator or other physical agitation device to promote aeration of material 38 prior to injection into balloon 12.

Other arrangements of inner ends 42 and 46 may be provided which are tailored to the physical characteristics of the specific material 38 used and may not necessarily be arranged as shown in FIG. 2. The disposition of material 38 within balloon 12 may be aided by the injection of gas through back pressure device 18 as gas and material 38 are injected through nozzle end 52. Back pressure within balloon 12 may assist in dispersing material 38 more uniformly about inner surface 14 so that the adhesive coating within balloon 12 is consistently coated with material 38. The timing of gas flow through injector 16 and back pressure device 18 may coordinated as required by the characteristics of the material being injected, the nature of the adhesive coating, and the size and shape of balloon 12 to ensure such uniform coating. Balloon 12 may also be rotated within apparatus 10 to aid in the uniform distribution of material 38 on inner surface 14.

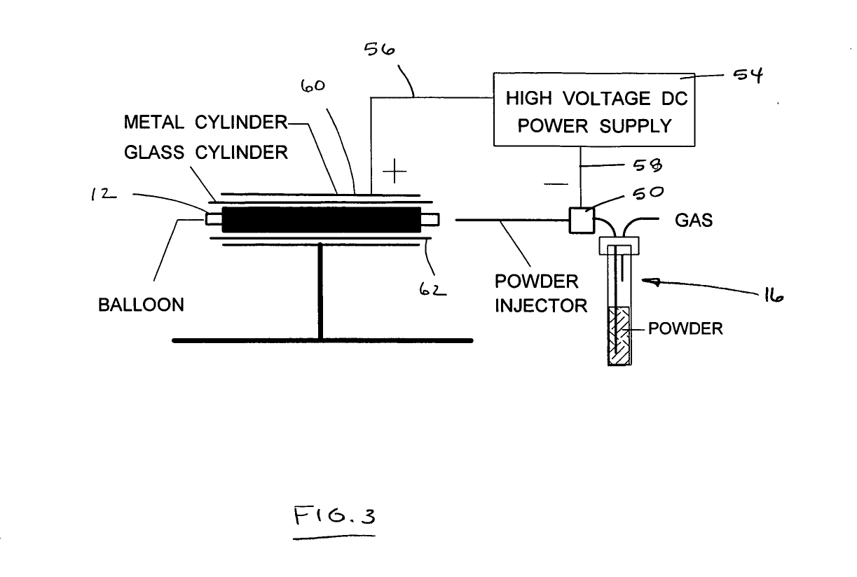

It is anticipated that some materials 38 may require more than mere physical agitation and the use of back pressure to uniformly coat inner surface 14. It may be desirable to provide corresponding electrostatic charges to the powder and to balloon 12 to aid in the positioning of material 38 within balloon 12. Such as arrangement is shown in FIG. 3, including a direct current power supply 54 with a positive electrical connection 56 being applied about balloon 12 and a negative electrical connection 58 being applied about material 38 at outer end 50 of injector 16. In this electrostatic configuration, a charge is not applied directly balloon 12 but to a metal cylinder 60 positioned about balloon 12. In addition, a second glass cylinder 62 is positioned between balloon 12 and metal cylinder 60. Positive connection 56 is electrically connected to metal cylinder 60 which will in turn provide an appropriate charge within balloon 12. Material 38 passes through outer end 50 which is electrically connected by negative connection 58 and is oppositely charged prior to injection into balloon 12. Once charged material 38 is injected within balloon 12, the opposite charges applied to material 38 and about balloon 12 will attract material 38 to inner surface 14.

Uniform coating of inner surface 14 with material 38 may be accomplished by any or a combination of the above described approaches, including injecting gas to provide back pressure through an opposite end of balloon 12, movement of nozzle 52 within balloon 12 as material 38 is injected, rotation of balloon 12 as material is injected, and electrostatic charges applied to material 38 and about balloon 12. The extent and nature of the approaches or combinations of approaches used or required may vary with the characteristics of the balloon and radiopaque coating material used. Any combination of approaches or sole use of any of the approaches is anticipated within the scope of the present disclosure.

Once inner surface 14 of balloon 12 has been coated as desired by apparatus 10, areas of inner surface 14 may be cleaned of material 38 adjacent to ends 20 and 22. Balloon 12 will eventually be sealed to provide an expandable arrangement for an angioplasty catheter. Such sealing may be carried out by heat sealing, ultrasonic welding, or any other appropriate sealing technique. It may be desirable that the portions of inner surface 14 that will be sealed are free of any material 38 or adhesive coating to ensure a good seal. So, after balloon 12 is removed from apparatus 10, ends 20 and 22 may be dipped into a bath of solvent to remove any adhesive coating or material 38 prior to curing the adhesive and securing material 38 to inner surface 14. It is also desirable that excess material 38 be removed from balloon 12 prior to curing, such as by blowing or washing.

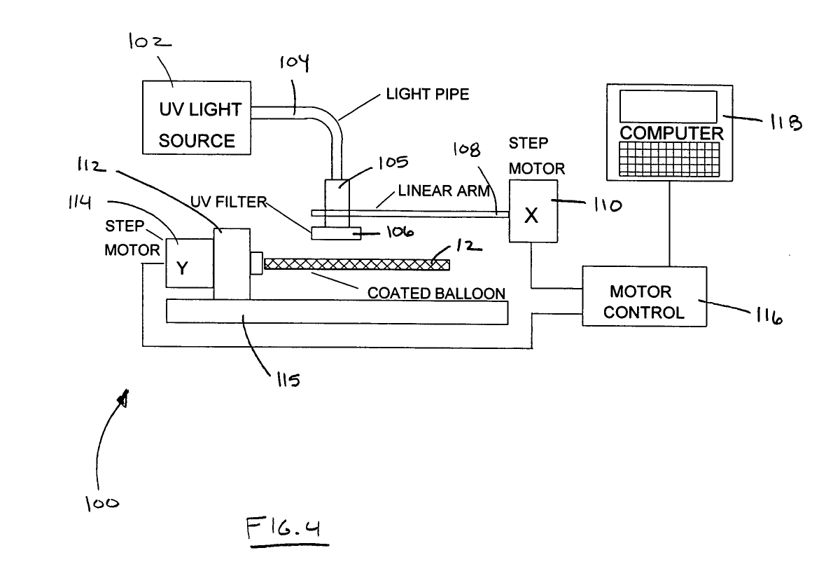

A curing device 100 is shown in FIG. 4, for curing the adhesive coating on inner surface 14 to fix radiopaque material 38 within balloon 12. As shown, curing device 100 includes a UV light source 102 and would thus be appropriate for use with a UV or light cured adhesive. It is anticipated that adhesives which are sensitive to and cured by other sources of energy, such as heat, may also be used on balloon 12, and that curing device 100 may be adapted to provide the appropriate energy source to cure these adhesives as well. It is also anticipated that coating apparatus 10 may be used with air curing or other self curing adhesives, so that a separate curing device is not required to fix material 38 to inner surface 14.

Curing device 100 includes a light pipe 104, such as an optical fiber pathway or other light transmission conduit from light source 102 to a head 105. Head 105 directs light from light source 102 against balloon 12 mounted to curing device 100. A specific filter 106 may be provided in head 105 to ensure that the light which will be most effective at curing the adhesive within balloon 12 is used. Different adhesives which may be used with balloon 12 may be sensitive to different portions of the spectrum of light generated by light source 102 and other portions of the spectrum may be filtered out. As shown, the adhesive in balloon 12 is a UV-sensitive adhesive and a UV specific light source 102 and filter 106 are included in curing device 100. If light source 102 is adapted to provide a very precisely tailored light to head 105, or the adhesive is sensitive to a broader range of the spectrum generated by light source 102, filter 106 may be not needed or included in curing device 100.

As shown, head 105 is mounted to a movable arm 108 which is in turn connected to a motor 110. Motor 110 is shown as a step motor but other motors or actuators may be used to move head 105 along a length of balloon 12. Balloon 12 is mounted about a spindle or shaft 112. Shaft 112 is in turn connected to a motor 114 and a platen or base 115. Motor 114 is shown as a step motor but other motors or actuators may be used to rotate shaft 112 so that all portions of a circumference of balloon 12 are exposed to light from head 105. Alternatively, motor 110 could be connected to base 115, with head 105 fixed in position within curing device 100. Motor 110 could then move balloon 12 back and forth under a fixed head 105 to expose balloon 12 to light from head 105.

Each of motors 110 and 114 are connected to a motor control 116, which is connected to and controlled by a computer 118. Computer 118 may signal motor control 116 to actuate motors 110 and 114 as needed to ensure that all of balloon 12 is adequately exposed to light or other energy from head 105 to sufficiently cure the adhesive along inner surface 14. Curing the adhesive will fix radiopaque material 38 to inner surface 14. A rate of rotation of spindle 112 and balloon 12, and a rate of movement of head 105 longitudinally along balloon 12 are selected according to the amount of light needed to cure the amount of adhesive within balloon 12 and are selected to ensure that excessive energy is not applied to balloon 12 to avoid damaging the balloon during curing. Balloon 12 may be made of a material which is sensitive to the light or energy used to cure the adhesive. Computer 118 will preferably control the time of exposure over all portions of balloon 12 to light or energy from head 105 to avoid any deleterious effects to balloon 12 while still ensuring curing of the adhesive within balloon 12. After curing, any remaining loose powder within balloon 12 can be washed away prior to sealing balloon 12 for use with an angioplasty catheter.

It is desirable that material 38 be applied along inner surface 14 of balloon 12, rather than on an external surface of balloon 12. While such coating could be more easily affected on an outer surface of balloon 12, such exterior coating may be susceptible to peeling off in the patient's blood stream, possibly causing a stroke or an infarct.

Providing a balloon 12 with a radiopaque coating permits fluids other than radiopaque fluids to be used to inflate or expand balloon 12 within a patient's bloodstream to perform angioplasty. Typically, liquids are used as the fluid as liquids are generally less compressible than air or other gases that might be used, and accidental releases of air from an angioplasty catheter could have devastating consequences for a patient. As balloon 12 is radiopaque by itself, without any injection of a radiopaque fluid, a neutral fluid such as common saline solution may be used to inflate the balloon. Saline is much less viscous that known or common radiopaque fluids which have been used in conventional balloon catheters permitting a smaller diameter fluid injection lumen to be used. Smaller fluid injection lumens mean that smaller diameter catheters may used for angioplasty. Smaller diameter catheters may mean less trauma to the patient being treated and may allow smaller vessels within a patient's vasculature to be treated.

While the above description has focused on securing a radiopaque material within a balloon, other imagable materials may be coated within the balloon, according to the present disclosure. Such alternative imagable materials might be visible using other known or to be developed corpal imaging techniques or apparatus, such as MRI, CAT Scans, PET Scans, or other approaches to imaging patients during a medical procedure. For radiopaque materials, metallic elements or alloys may be used to make the coating radiopaque. Also, while the above description is directed to production of a balloon for angioplasty, balloon catheters for other uses within a patient are also anticipated according to the present disclosure.

The embodiments of the inventions disclosed herein have been discussed for the purpose of familiarizing the reader with novel aspects of the present invention. Although preferred embodiments have been shown and described, many changes, modifications, and substitutions may be made by one having skill in the art without unnecessarily departing from the spirit and scope of the present invention. Having described preferred aspects and embodiments of the present invention, modifications and equivalents of the disclosed concepts may readily occur to one skilled in the art. However, it is intended that such modifications and equivalents be included within the scope of the claims which are appended hereto.

What is claimed is:

1. A method of making a remotely imagable balloon for mounting on a catheter, the method comprising:
injecting an adhesive and an imagable material within the balloon along an inner surface of the balloon.

2. The method of claim 1, further comprising first injecting the adhesive within the balloon along the inner surface of the balloon and then injecting the imagable material within the balloon onto the adhesive.

3. The method of claim 1, wherein the imageability of the imagable material is radiopacity.

4. The method of claim 1, wherein the imagable material is a metal.

5. The method of claim 1, wherein the adhesive is a light curing adhesive, and further comprising fixing the imagable material to the inner surface of the balloon by exposing the adhesive to light and curing the adhesive.

6. The method of claim 1, wherein the adhesive is one of epoxy, urethane, silicone, acrylic, or cyanoacrylate.

7. The method of claim 1, wherein the adhesive is first diluted with a solvent prior to injecting the adhesive along the inner surface of the balloon.

8. The method of claim 7, wherein the solvent is one of acetone, alcohol, freon, or a combination of these solvents.

9. The method of claim 1, further comprising the application of an electrostatic charge to distribute the imagable material along the inner surface of the balloon.

10. The method of claim 1, further comprising injection of the imagable material through a first end of the balloon and injection of a gas into a second opposite end of the balloon to alter the distribution of the imagable material.

11. The method of claim 1, further comprising injection of the imagable material through a first end and a second opposite end of the balloon to distribute the material on the inner surface of the balloon.

12. The method of claim 1, further comprising agitating the imagable material prior to injection of the imagable material along the inner surface of the balloon.

13. The method of claim 1, further comprising warming the imagable material to reduce moisture content of the imagable material prior to injection of the imagable material along the inner surface of the balloon.

14. The method of claim 5, further comprising cleaning the inner surface adjacent an open end before the adhesive is cured.

15. The method of claim 14, wherein the inner surface of the balloon adjacent the open end is cleaned with a solvent.

16. A powder injector used to produce an imagable balloon comprising:
a gas inlet in fluid communication with a powder vessel, the powder vessel including a powdered imagable material, the gas inlet connected with a gas source;
a gas outlet in fluid communication with the powder vessel;
an expulsion needle connected to an outer end of the gas outlet and sized to fit within a balloon to be coated with the imagable material;
the powder vessel including a powder dryer; and,
the gas outlet including an inner end positioned within the powder vessel so that gas within the powder vessel and imagable material borne by the gas are carried from the powder vessel through the expulsion needle into the balloon.

17. The injector of claim 16, wherein the expulsion needle is 18 to 25 gauge.

18. The injector of claim 16, further comprising an agitation means to aerate the material within the powder vessel.

19. A powder injector used to produce an imagable balloon comprising:
a gas inlet in fluid communication with a powder vessel, the powder vessel including a powdered imagable material, the gas inlet connected with a gas source;
a gas outlet in fluid communication with the powder vessel;
an expulsion needle connected to an outer end of the gas outlet and sized to fit within a balloon to be coated with the imagable material;
an agitation means to aerate the material within the powder vessel; and,
the gas outlet including an inner end positioned within the powder vessel so that gas within the powder vessel and imagable material borne by the gas are carried from the powder vessel through the expulsion needle into the balloon.

20. An adhesive curing station for curing adhesive applied to coated catheter balloons, the station comprising:
a light source emitting light;
a spindle onto which the light is directed;
a hollow form being positioned about the spindle, the hollow form having an inner surface coated with a light curable adhesive, the hollow form being transparent to at least a portion of the light emitted by the light source; and,
a mechanism to scan at least a portion of the light emitted from the light source along the hollow form to cure the adhesive within the hollow form without causing heat damage to the hollow form.

21. The adhesive curing station of claim 20, wherein the hollow form is a balloon for use with a catheter.

22. The adhesive curing station of claim 20, wherein the light source emits UV light and the adhesive within the hollow form is a UV-cured adhesive.
